(19) United States
(12) Patent Application Publication
(10) Pub. No.: US 2001/0046662 A1
(43) Pub. Date: Nov. 29, 2001

(54) Method of inactivating pathogens in a red blood cell-containing composition

(75) Inventors: Stephen J. Wagner, Columbia, MD (US); Andrey Skripchenko, Gaithersburg, MD (US)

Correspondence Address: STERNE, KESSLER, GOLDSTEIN & FOX P.L.L.C., Attorneys at Law, Suite 600, 1100 New York Avenue, N.W., Washington, DC, 20005-3934, US

(73) Assignee: The American National Red Cross

(21) Appl. No.: 09/774,608

(22) Filed: Feb. 1, 2001

Related U.S. Application Data
(60) Provisional application No. 60/196,484, filed on Apr. 12, 2000.

(51) Int. Cl. C12N 5/08

(52) U.S. Cl. 435/2

(57) ABSTRACT

The invention provides a method for reducing the level of active pathogenic contaminants, such as viruses, bacteria and parasites, found in whole blood and blood components. The method comprises adding to a red blood cell-containing composition at least one phenothiazin-5-ium dye in an amount effective to reduce the level of active pathogenic contaminants and at least one blocking compound in an amount effective to enhance pathogen inactivation and protect red blood cells from hemolysis, and irradiating the red blood cell-containing composition with light of an appropriate intensity and wavelength for a time sufficient to reduce the level of active pathogenic contaminants in the red blood cell-containing composition. An irradiated red blood cell-containing composition comprising red blood cells, a phenothiazin-5-ium dye and a blocking compound is also provided.

### CROSS-REFERENCE TO RELATED APPLICATIONS

This application claims the benefit of the filing date of U.S. Provisional Application No. 60/196,484, filed Apr. 12, 2000, which is herein incorporated by reference in its entirety.

### BACKGROUND OF THE INVENTION

1. Field of the Invention

The present invention is directed to methods and compounds useful for reducing the level of active pathogenic contaminants, such as viruses, bacteria and parasites, found in whole blood and blood components.

2. Background Art

Among the risks inherent in handling or being transfused with blood, blood proteins or other blood components is the risk of infection from pathogenic contaminants, including, e.g., human immunodeficiency virus (HIV), serum hepatitis, cytomegalovirus, Epstein-Barr virus, herpes simplex, infectious mononucleosis, syphilis and malaria. Virucidal methods, including heat, solvent-detergent and gamma irradiation, have been used to produce non-infectious plasma derivatives, but such methods are generally ineffective or too harsh to be routinely used for effective decontamination of whole blood, red cells and/or platelets. Indeed, any virucidal treatment that damages or introduces harmful or undesirable contaminants into the product is unsuitable for decontamination of a product intended for transfusion into an animal, particularly a human. Due to the critical need for transfusable red blood cells, it is of great importance to develop efficient methods that can be readily used to decontaminate cellular blood components and whole blood without substantially or irreversibly altering or harming them.

Efficient decontamination treatments that inactivate contaminating pathogens but do not harm the cellular fractions of blood are not readily available.

Common decontamination treatments include the use of photosensitizers, which in the presence of dissolved oxygen and upon exposure to light that includes wavelengths absorbed by the photosensitizer, inactivate viruses (see EP 0 196 515). Typically such photochemicals are dyes or other compounds that readily absorb UV or visible light in the presence of dissolved oxygen. These compounds include merocyanine 540 (“MC 540”) (see U.S. Pat. No. 4,775,625), porphyrin derivatives (see U.S. Pat. Nos. 5,536,238 and 4,878,891) as well as other photosensitizers.

Red blood cell decontamination methods using such photochemicals have previously encountered a problem due to the absorbency, by hemoglobin, of light at wavelengths necessary to activate such compounds. As such, increased virucidal activity of these compounds is realized when the absorption spectrum of the photosensitizer does not significantly overlap the absorption spectra of pigments present in the blood, such as hemoglobin

It is well known that viruses, such as hepatitis or HIV virus, may be resident within human blood. The viruses residing in blood may be “intracellular,” i.e., contained within one of the cellular components of blood, such as white blood cells, or they may be “extracellular,” i.e., freely existing in the plasma. Regardless of where the virus resides, the presence of the virus in the bloodstream poses the risk of infection and disease not only to the host, but also, if the blood or a blood component is collected and transfused, to the recipient. It is therefore preferable if the photodynamic treatment inactivates both extracellular and intracellular viruses, including intracellular forms such as proviruses.

In order to minimize cellular damage, it is preferable that the photosensitizer be non-toxic to cellular blood components and selectively bind to a component of the virus either that is not present in red cells or platelets or, if present therein, that is not essential to red cells' or platelets' function. For example, because blood platelets, erythrocytes and plasma proteins do not contain genomic nucleic acids, compounds that specifically target nucleic acids would desirably result in the targeting of viruses. White blood cells do contain nucleic acids and, therefore, will also be targeted by these compounds. However, since many of these white blood cells may be infected with unwanted viruses and white blood cells are typically filtered from blood products before being administered to patients, the targeting of white blood cells by such compounds is not undesirable. It is further preferable that the virucidal activity of the photosensitizer be uninhibited by the presence of plasma proteins, such as coagulation proteins, albumin and the like.

Treatment with known photochemicals, however, frequently does damage cellular blood components. For example, photochemicals such as the porphyrins (see U.S. Pat. No. 4,878,891) and MC 540 (see U.S. Pat. No. 4,775,625) cause membrane damage in the presence of light and oxygen, which significantly reduces the survivability of the phototreated red cells during storage. Similarly, treatment of red blood cells using phthalocyanine 4 with type 1/type 2 quenchers caused red cell damage even under optimized conditions—about 2% of the cells hemolyze after 21 days of storage (the current FDA guideline for hemolysis is <1% after 6 weeks of storage at 1-6° C.) (Vyas, Transfusion 35:367-70 (1995)).

Additionally, both MC 540 and porphyrin derivatives apparently bind to blood components, such as albumin (Lagerberg et al., Biochim. Biophys. Acta 1235:428-436 (1995); Transfusion 29:42S (1989)). For example, the effect of MC 540 on platelets and the influence of albumin on MC 540's virucidal activity has been studied. In the presence of light and MC 540, the platelets aggregated. Albumin, however, prevented aggregation and inhibited the inactivation of viral contaminants by MC 540 plus light. Similarly, because of such competitive inhibition reactions with blood and/or plasma components, some dyes are not suitable for decontaminating blood, cellular blood components, or any blood-derived products containing high plasma concentrations (as plasma concentration increases, the percentage of viral inactivation substantially decreases).

The phenothiazin-5-ium dyes such as methylene blue, toluidine blue O, thionine, azure A, azure B, and azure C have been shown to inactivate animal viruses (see U.S. Pat. Nos. 4,407,282, 4,402,318, 4,305,390 and 4,181,128). Light-induced activation of photochemical agents such as methylene blue is believed to result in the production of singlet oxygen which enhances the reactions that inactivate virus. One target for virus inactivation is viral nucleic acids (Abe et al., Photochem. Photobiol. 61:402-409 (1995)). Methylene blue and visible light damage guanine residues of nucleic acids (Simon et al., J. Mol. Biol. 4:488-499 (1962)), and methylene blue and white light produce 8-hydroxy-guanine in DNA (Floyd et al., Arch. Biochim. Biophys. 273:106-111 (1989)). Based on this activity, phenothiazin-5-ium dyes have been employed for inactivation of extracellular enveloped viruses in blood and blood components because the dyes absorb light at wavelengths that are not substantially absorbed by hemoglobin (see U.S. Pat. Nos. 6,030,767 and 5,545,516).

These particular phenothiazin-5-ium dyes, however, have certain drawbacks that limit their usefulness for inactivating pathogens in whole blood or blood components. For example, red cells readily take up or bind such dyes (Sass et al., J. Lab. Clin. Med. 73:744-752 (1969)). In addition, photosensitized oxidation of biological membranes is deleterious to membrane structure and function (methylene blue cross-links the membrane protein, spectrin, in red cells exposed to visible light and oxygen) (Girotti, Biochim. Biophys. Acta. 602:45-56 (1980)). Also, methylene blue treated red blood cells have been shown to bind to plasma proteins, such as IgG and albumin (Wagner et al., Transfusion 33:30-36 (1992)).

Despite careful donor selection and extensive laboratory testing, a small risk of pathogen transmission by transfusion still exists. While the prior art methods represent progress in the inactivation of pathogenic contaminants, such as viruses, in biological fluids, such as blood or blood components, there is still room for improvements. There remains an acute need for an efficient, safe, effective, reproducible and rapid method for reducing the level of active pathogenic contaminants, particularly HIV and hepatitis, in whole blood or blood components without rendering the blood or blood components unsuitable for transfusion.

### BRIEF SUMMARY OF THE INVENTION

Accordingly, the present invention provides a method of reducing the level of both intracellular and extracellular active pathogenic contaminants present in red blood cell-containing compositions under conditions that will neither harm the recipient nor the in vitro/in vivo properties of the red blood cells.

In one embodiment, the present invention is directed to a method of reducing the level of active pathogenic contaminants present in a red blood cell-containing composition which comprises adding to the red blood cell-containing composition at least one phenothiazin-5-ium dye in an amount effective to reduce the level of active pathogenic contaminants and at least one blocking compound in an amount effective to enhance pathogen inactivation and protect red blood cells from hemolysis, and irradiating the red blood cell-containing composition with light of an appropriate intensity and wavelength for a time sufficient to reduce the level of active pathogenic contaminants in the red blood cell-containing composition.

A further embodiment of the present invention is directed to an irradiated red blood cell-containing composition which comprises red blood cells, a phenothiazin-5-ium dye and a blocking compound.

Other features and advantages of the present invention will be set forth in the detailed description of preferred embodiments that follows, and in part will be apparent from the description or may be learned by practice of the invention. These advantages of the invention will be realized and attained by the methods particularly pointed out in the written description and claims hereof.

It is to be understood that both the foregoing general description and the following detailed description are exemplary and explanatory only and are intended to provide further explanation of the invention as claimed.

### BRIEF DESCRIPTION OF THE DRAWINGS/FIGURES

FIG. 1 is a depiction of the effects of quinacrine on the photoinactivation of intracellular and extracellular vesicular stomatitis virus in a red blood cell-containing mixture (45% hematocrit) suspended in RAS-2 (Erythrosol) using 1,9-dimethylmethylene blue.

FIG. 2 is a depiction of the effects of quinacrine on the hemolysis of 1,9-dimethylmethylene blue and light treated red blood cells after six weeks of storage at 1-6° C.

FIG. 3 is a depiction of the effects of riboflavin on the photoinactivation of extracellular vesicular stomatitis virus in a red blood cell-containing mixture (45% hematocrit) suspended in RAS-2 (Erythrosol) using 5 μM of 1,9-dimethylmethylene blue.

### DETAILED DESCRIPTION OF THE INVENTION

Definitions

Unless defined otherwise, all technical and scientific terms used herein are intended to have the same meaning as is commonly understood by one of ordinary skill in the relevant art. All patents and publications mentioned herein are expressly incorporated by reference.

As used herein, the term “pathogenic contaminant of a red blood cell-containing composition” is intended to mean a contaminant that, upon handling or transfusion into a recipient may cause disease in the handler or recipient, respectively.

As used herein, the term “pathogen” is intended to mean any replicable agent that can be found in or infect a red blood cell-containing composition.

Such pathogens include the various viruses, bacteria and parasites known to those of skill in the art to generally be found in or infect whole blood or blood components and other pathogenic contaminants not yet known. Illustrative examples of such pathogens include, but are not limited to: bacteria, such as Streptococcus species, Escherichia species and Bacillus species; viruses, such as human immunodeficiency viruses and other retroviruses, herpes viruses, paramyxoviruses, cytomegaloviruses, hepatitis viruses (including hepatitis A, hepatitis B, and hepatitis C), pox viruses and toga viruses; and parasites, such as malarial parasites, including Plasmodium species, and trypanosomal parasites.

As used herein, the term “red blood cell-containing composition” means whole blood, red blood cell concentrates and any other composition that contains red blood cells. Other than red blood cells, the composition can also contain a biologically compatible solution, such as ARC-8, Nutricell (AS-3), ADSOL (AS-1), Optisol (AS-5) or RAS-2 (Erythrosol), and one or more cellular blood components, one or more blood proteins, or a mixture of one or more cellular blood components and/or one or more blood proteins. Such compositions may also contain a liquid blood component, such as plasma.

As used herein, the term “blood components” is intended to mean one or more of the components that may be separated from whole blood and include, but are not limited to: cellular blood components, such as red blood cells and platelets; blood proteins, such as blood clotting factors, enzymes, albumin, plasminogen, and immunoglobulins; and liquid blood components, such as plasma and plasma-containing compositions.

As used herein, the term “cellular blood component” is intended to mean one or more of the components of whole blood that comprises cells, such as red blood cells or platelets.

As used herein, the term “blood protein” is intended to mean one or more of the proteins that are normally found in whole blood. Illustrative examples of blood proteins found in mammals (including humans) include, but are not limited to, coagulation proteins (both vitamin K-dependent, such as Factor VII or Factor IX, and non-vitamin K-dependent, such as Factor VIII and von Willebrands factor), albumin, lipoproteins (high density lipoproteins and/or low density lipoproteins), complement proteins, globulins (such as immunoglobulins IgA, IgM, IgG and IgE), and the like.

As used herein, the term “liquid blood component” is intended to mean one or more of the fluid, non-cellular components of whole blood, such as plasma (the fluid, non-cellular portion of the blood of humans or animals as found prior to coagulation) or serum (the fluid, non-cellular portion of the blood of humans or animals after coagulation).

As used herein, the term “blocking compound” is intended to mean a substance which, at appropriate concentrations, can enhance pathogen inactivation, as compared to phenothiazin-5-ium dye phototreatment without the substance, and/or reduce the red blood cell membrane damaging effects, such as hemolysis, from phototreated red blood cells.

In general, the blocking compound should not be capable of producing singlet oxygen or other reactive oxygen species, or cannot produce singlet oxygen or other reactive oxygen species if irradiated with red light (it may absorb light in a different region of the spectrum). Otherwise, the red cells would be damaged by the photodynamic action of the blocking compound. In addition, the blocking compound should bind to nucleic acid more poorly than the red light-activated phenothiazin-5-ium dye (so as not to inhibit binding of the red-light activated phenothiazin-5-ium dye and thereby inhibit virus inactivation).

Structurally, the blocking compound should contain a fused aromatic ring system containing at least three rings, where at least one of the rings contains at least one heteroatom, such as nitrogen, oxygen or sulphur. Examples of such moieties include, but are not limited to, an acridine moiety, an alloxazine moiety, a phenothiazine moiety, a phenoxazine moiety and a phenazine moiety. Examples of blocking compounds of the present invention which include one of these moieties include, but are not limited to, quinacrine, riboflavin and lumichrome, and derivatives thereof.

Compositions containing cellular blood components, in addition to red cells, and/or a blood protein may optionally be leukodepleted. As used herein, the term “leukodepleted” is intended to mean that the concentration of leukocytes in the composition has been reduced by a specified amount, such as a factor of 10⁵. It is not necessary that compositions be leukodepleted before application of the methods of the present invention.

As used herein, the term “transfusable composition” is intended to mean a composition that can be transfused into the blood stream of a mammal. Transfusable compositions can contain whole blood, one or more blood components, such as one or more cellular blood components, one or more blood proteins, and one or more liquid blood components, or mixtures of whole blood and one or more blood components, such as red blood cells, clotting factors or plasma.

As used herein, the term “decontamination” is intended to mean a process whereby the level of active pathogens, such as viral or bacterial contaminants, in a given composition is reduced. Such reduction may occur by rendering the pathogens inactive and/or noninfectious or by reducing the number of pathogens in the composition. A composition containing whole blood or a blood component that has been “decontaminated” can be transfused or manipulated without harming or infecting anyone exposed thereto.

The ratio of the titer of the control sample to the titer of virus in each of the treated samples is a measure of viral inactivation. As used herein, the term “log₁₀ inactivation” is intended to mean the log₁₀ of this ratio. Typically, a log₁₀ inactivation of at least about 4 indicates that the treated sample has been decontaminated.

As used herein, the term “fluence” is intended to mean a measure of the energy per unit area of sample and is typically measured in joules/cm² (J/cm²). As used herein, the term “fluence rate” is intended to mean a measure of the amount of energy that strikes a given area of a sample in a given period of time and is typically measured as milliwatts (mW)/cm² or as joules/cm² per unit time of exposure.

As used herein, the term “appropriate wavelength and intensity” is intended to mean light of a wavelength and intensity that can be absorbed by the dye, but does not substantially damage the blood or blood components present. It is well within the level of skill in the art to select such wavelength and intensity empirically based on certain relevant parameters, such as the particular dye employed and its concentration in the composition.

As used herein, the term “phenothiazin-5-ium dye” is intended to mean a compound having the general structure

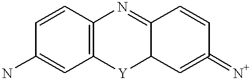

which is soluble in aqueous solutions and also capable in sufficient amounts to reduce the level of active pathogenic contaminants in red blood cell-containing compositions upon irradiation with light of a suitable intensity and wavelength.

This class of compounds includes, but is not limited to, 1,9-dimethylmethylene blue, methylene blue, toluidine blue O, thionine, azure A, azure B and azure C. The unspecified valences of the carbon and nitrogen atoms in the formula above can be occupied by hydrogen atoms or by any organic or inorganic moiety which does not adversely affect the ability of the dye to reduce the level of active pathogens in a red blood cell-containing composition when irradiated with light of an appropriate intensity and wavelength. One skilled in the art can determine the suitability of a particular substituent group or groups empirically using any of the standard assays for determining the level of active intracellular or extracellular pathogenic contaminants.

Illustrative examples of organic moieties include, but are not limited to, alkyl groups, alkenyl groups, alkynyl groups, hydroxy groups, methoxy groups, alkoxy groups, aryl groups, heteroaryl groups, aryloxy groups, heteroaryloxy groups, nitro groups, amine groups, amide groups, alkoxycarbonyl groups, alkylcarboxyl groups, arylcarboxyl groups, aralkyl groups, cyano groups, azide groups, haloalkyl groups, haloaryl groups. Preferable organic moieties include alkyl groups, such as methyl, ethyl, and propyl, alkenyl groups, such as ethenyl, alkynyl groups, such as acetenyl, and amine groups, such as monomethylamine and dimethylamine.

Illustrative examples of suitable inorganic moieties include, but are not limited to, sulfur, selenium and tellurium. Preferred inorganic moieties include sulfur and selenium.

The preferred phenothiazin-5-ium dyes employed in the present invention include those of the formula:

[chemical structure]

wherein

each of R¹, R¹′, R₂, and R₂ is independently selected from the group consisting of a methyl group, an ethyl group, a propyl group, an alkenyl group, an alkynyl group, a nitrile, an aralkyl group, a hydroxy group, an alkoxy group, an amine group, and a hydrogen atom,

each of R₃, R₃′, R₄, R₄′, R₅ and R₅′ is independently selected from the group consisting of an alkyl group, an alkenyl group, an alkynyl group, a nitrile, an azide, an aryl group, an aralkyl group, a heteroaryl group, an alkoxy group, an aryloxy group, an amine group, and a hydrogen atom, or

any two of R₁, R₁′, R₂, R₂′, R₃, R₃′, R₄, R₄′, R₅ and R₅′ together form an aryl or heteroaryl ring;

Y is selected from the group consisting of S, Se and Te; and

X is a counter-ion;

with the provisos:

(i) that if each of R₁, R₁′, R₂ and R₂′ is independently a hydrogen atom or an alkyl group, then at least one of R₃, R₃′, R₄, R₄′, R₅ and R₅′ is other than a hydrogen atom,

(ii) that if one of R₄ and R₄′ is an alkyl group, then at least one other of R₃, R₃′, R₄, R₄′, R₅ and R₅′ is other than a hydrogen atom, and

(iii) that if any two of R₃, R₃′, R₄, R₄′, R₅ and R₅′ together form an aryl or heteroaryl ring, each of R₁, R₁′, R₂, R₂′ is other than a hydrogen atom.

As used herein, the term “effective amount of phenothiazin-5-ium dye” is intended to mean an amount sufficient to provide a concentration of dye in the red blood cell-containing composition that is acceptable for transfusion and which is effective to reduce the level of active pathogens in the composition when irradiated with light of an appropriate intensity and wavelength.

As used herein, the term “effective amount of blocking compound” is intended to mean an amount sufficient to provide a concentration of the compound in the red blood cell-containing composition that is acceptable for transfusion and which is effective to increase pathogen inactivation, as compared to phenothiazin-5-ium dye phototreatment without the compound, and/or to reduce the level of hemolysis from phototreated red blood cells, as compared to hemolysis levels of phenothiazin-5-ium dye phototreatment without the compound.

As used herein, the term “biologically compatible solution” is intended to mean an aqueous solution in which cellular blood components can be exposed or stored, such as by being suspended therein, and remain viable, i.e., retain their essential biological and physiological characteristics. Such biologically compatible solutions preferably contain an effective amount of at least one anticoagulant.

As used herein, the term “biologically compatible buffered solution” is intended to mean a biologically compatible solution having a pH and osmotic properties (e.g., tonicity, osmolality and/or oncotic pressure) suitable for maintaining the integrity of the cell membrane of cellular blood components. Suitable biologically compatible buffered solutions typically have a pH between 5.0 and 8.5 and are isotonic or only moderately hypotonic or hypertonic. Biologically compatible buffered solutions are known and readily available to those of skill in the art. Illustrative examples of suitable solutions include, but are not limited to, Nutricell, ADSOL, ARC-8 and RAS-2 (Erythrosol).

Preferred Embodiments

In one aspect of the invention there is provided a method of reducing the level of active pathogenic contaminants present in a red blood cell-containing composition which comprises adding to the red blood cell-containing composition at least one phenothiazin-5-ium dye in an amount effective to reduce the level of active pathogenic contaminants and at least one blocking compound in an amount effective to enhance pathogen inactivation and protect red blood cells from hemolysis, and irradiating the red blood cell-containing composition with light of an appropriate intensity and wavelength for a time sufficient to reduce the level of said active pathogenic contaminants in the red blood cell-containing composition.

As a first step when practicing any of the embodiments of the invention disclosed herein, whole blood is preferably drawn from a donor into a suitable biologically compatible buffered solution containing an effective amount of at least one anticoagulant. Suitable anticoagulants are known to those skilled in the art, and include, but are not limited to, lithium, potassium or sodium oxalate (15 to 25 mg/10 mL blood), sodium citrate (40 to 60 mg/10 mL blood), heparin sodium (2 mg/10 mL blood), disodium EDTA (10 to 30 mg/10 mL whole blood), ACD-Formula A solution (1.0 mL/10 mL blood) and citrate-phosphate-dextrose solution (CPD) (1.0 mL/10 mL blood).

The whole blood so collected can then be decontaminated according to the methods of the present invention. Alternatively, the whole blood can first be separated into blood components, including, but not limited to, plasma, platelets and red blood cells, by any method known to those of skill in the art.

For example, the blood can be centrifuged for a sufficient time and at a sufficient centrifugal force to sediment or pack the red blood cells. Leukocytes collect primarily at the interface of the red cells and the plasma-containing supernatant in the buffy coat region. The supernatant, which contains plasma, platelets, and other blood components, can then be removed and centrifuged at a higher centrifugal force, whereby the platelets sediment.

Human blood normally contains about 7×10⁹ leukocytes per liter. The concentration of leukocytes, which form a layer on top of the sedimented red cells, can be decreased by filtering whole blood prior to centrifugation through a filter that decreases their concentration by several orders of magnitude. Leukocytes can also be removed from each of the components by filtration through an appropriate filter that removes them from the solution.

In a preferred embodiment of this invention, the red blood cell-containing composition to be decontaminated is obtained in, prepared in or introduced into gas permeable blood preservation bags which are sealed and flattened to a width sufficiently narrow to permit light to irradiate the contents, such that any pathogenic contaminant present in the red blood cell-containing composition in the bag will be irradiated. Preferably, the red blood cell-containing composition to be decontaminated is obtained in, prepared in or introduced into gas permeable blood preservation bags which are flattened to a width of about 4 mm or less thick, more preferably about 2 mm or less. Any such blood bag known to those of skill in the art can be used provided that the bag is transparent to the selected wavelength of light.

The method of the present invention can be practiced in a static system or in a flow system. Examples of systems which can be used in the present invention are disclosed, for example, in U.S. Pat. Nos. 5,304,113 and 5,030,200.

The red blood cell-containing composition that is to be decontaminated can also include any suitable biologically compatible buffered solution known to those of skill in the art. Examples of such buffers include, but are not limited to, ADSOL, ARC-8, Nutricell and RAS-2 (Erythrosol).

According to the inventive process, a blocking compound is added to the red blood cell-containing composition. It was unexpectedly found that pathogen inactivation can be enhanced and photoinduced red cell damage can be diminished by the addition of the blocking compound of the present invention.

In general, the blocking compound of the present invention should neither absorb red light nor produce reactive oxygen species upon red light illumination. Further, the blocking compound should bind to nucleic acid more poorly than the red light-activated phenothiazin-5-ium dye.

While not wishing to be bound by any theory of operability, it is believed that the blocking compound acts as a competitive inhibitor to prevent the phenothiazin-5-ium dye from binding to red blood cells, causing a higher effective phenothiazin-5-ium dye concentration available for pathogen inactivation. The red cell protection may be due to reduced phenothiazin-5-ium dye binding to blocking compound-bound red cells which would cause less light- driven, transient, reactive oxygen species from being generated near the red cell membrane. As such, the competitive inhibition of phenothiazin-5-ium binding rather than simple antioxidant activity may be responsible for the reduction of hemolysis. At any rate, whatever the mechanism by which the blocking compound of the present invention exerts its inactivation enhancement or red cell protection, it is this finding which is of practical importance.

Structurally, the blocking compound contains a fused aromatic ring system containing at least three rings, where at least one of the rings contains at least one heteroatom, such as nitrogen, oxygen or sulphur. Examples of such moieties include, but are not limited to, an acridine moiety, an alloxazine moiety, a phenothiazine moiety, a phenoxazine moiety and a phenazine moiety. Examples of blocking compounds of the present invention which include one of these moieties include, but are not limited to, quinacrine, riboflavin and lumichrome, and derivatives thereof. Any blocking compound can be used in the present invention provided that the compound enhances pathogen inactivation in the presence of a phenothiazin-5-ium dye and protects phototreated red blood cells from hemolysis.

In a preferred embodiment of the present invention, quinacrine is employed as the blocking compound. Quinacrine is utilized for the treatment of giardiasis and cestodiasis, and has also been used for the treatment and suppression of malaria. Further, quinacrine has been reported to be a phospholipase A₂ inhibitor in platelets.

Quinacrine is also known as mepacrine and is described by the following formulas: N⁴-(6-chloro-2-methoxy-9-acridinyl)-N¹,N¹-diethyl-1,4-pentanediamine dihydrochloride; 6-chloro-9-[[4-(diethylamino)-1-methylbutyl]amino]-2-methoxyacridine; and 3-chloro-7-methoxy-9-(l -methyl-4-diethyl aminobutylamino)acridine. Quinacrine is most commonly described in the literature in the form of its hydrochloride salt. It is available in formulations for pharmaceutical use under a number of trade designations, including Atabrine® Hydrochloride (Sanofi Winthrop Pharmaceuticals, New York, N.Y.). Any derivatives of quinacrine including, but not limited to, quinacrine hydrochloride, quinacrine dihydrochloride, and quinacrine dihydrochloride dihydrate, can be used in the present invention provided that the quinacrine derivative enhances pathogen inactivation in the presence of a phenothiazin-5-ium dye and protects phototreated red blood cells from hemolysis.

In another preferred embodiment of the present invention, riboflavin is employed as the blocking compound. Riboflavin (vitamin B₂) is a precursor of the coenzymes flavin adenine dinucleotide and flavin mononucleotide and is synthesized by plants and by most microorganisms, but not by higher animals. Riboflavin is also known as ovoflavin, lactoflavin, vitamin G, beflavin and flavaxin and is described by the following formulas: 7,8-dimethyl-10-(D-ribo2,3,4,5-tetrahydroxypentyl)isoalloxazine; and 7,8-dimethyl-10-ribitylisoalloxazine.

Riboflavin for use in the invention is readily available and can be prepared either chemically or microbially according to methods known to those skilled in the art. See, e.g., U.S. Pat. Nos. 5,925,538; 5,589,355; and 4,656,275. In addition, any derivative of riboflavin, including, but not limited to, riboflavin monophosphate, can be used in the present invention provided the derivative enhances pathogen inactivation in the presence of a phenothiazin-5-ium dye and/or protects phototreated red blood cells from hemolysis.

In yet another preferred embodiment of the invention, lumichrome is employed as the blocking compound. Lumichrome is described by the following formulas: 7,8-dimethylbenzo[g]pteridine-2,4-(1H,3H)-dione; and 7,8-dimethylalloxazine. Lumichrome, a common breakdown product of riboflavin, is produced from riboflavin by photodegradation in neutral or acidic solutions as well as by enzymes in bacteria and plants. Further, any derivative of lumichrome can be used in the present invention provided the derivative enhances pathogen inactivation in the presence of a phenothiazin-5-ium dye and/or protects phototreated red blood cells from hemolysis.

The effective concentration of the blocking compound to be used can be determined empirically by one of skill in the art. Any concentration can be used provided it is an amount sufficient to provide a concentration of the compound in the red blood cell-containing composition that is effective to increase pathogen inactivation, as compared to phenothiazin-5-ium dye phototreatment without the compound, and/or to reduce the level of hemolysis from phototreated red blood cells, as compared to hemolysis levels of phenothiazin-5-ium dye phototreatment without the compound. Preferably, the selected compound is non-toxic and the effective concentration is acceptable for transfusion so that the treated blood or blood component does not require additional manipulation to remove the compound.

For example, when quinacrine is used as the blocking compound, the effective amount is an amount sufficient to provide a concentration of quinacrine in the red blood cell-containing composition from about 250 μM to about 1000 μM, more preferably from about 350 μM to about 750 μM. When riboflavin is used as the blocking compound, the effective amount is an amount sufficient to provide a concentration of riboflavin in the red blood cell-containing composition from about 5 μM to about 45 μM, more preferably from about 15 μM to about 40 μM. When lumichrome is used as the blocking compound, the effective amount is an amount sufficient to provide a concentration of lumichrome in the red blood cell-containing composition from about 300 μM to about 3 mM. Further, in view of the fact that quinacrine, riboflavin and lumichrome are red blood cell blocking compounds, the optimal concentration of each compound would be expected to be hematocrit dependent.

In accordance with the present invention, an effective amount of at least one selected phenothiazin-5-ium dye is introduced into the red blood cell-containing composition. Any amount sufficient to provide a concentration of dye in the composition that is acceptable for transfusion and which is effective to reduce the level of active pathogens in the composition when irradiated with light of an appropriate intensity and wavelength can be used. Preferably, the selected dye is non-toxic and the effective concentration is acceptable for transfusion so that the treated blood or blood component does not require additional manipulation to remove the dye.

The effective concentration of dye to be used can be determined empirically by one of skill in the art. Preferably, the effective concentration of the dye is from about 0.2 μM to about 50 μM, more preferably from about 1 μM to about 25 μM.

The phenothiazin-5-ium dyes used in the invention can be prepared according to the methods and techniques known to those skilled in the art. Suitable synthetic methods are described, for example, in U.S. Pat. No. 4,962,197. Any phenothiazin-5-ium dye capable in sufficient amounts to reduce the level of active pathogenic contaminants in red blood cell-containing compositions upon irradiation with light of a suitable intensity and wavelength can be used in the present invention. Preferably, the phenothiazin-5-ium dye has the formula disclosed in U.S. Pat. No. 6,030,767. In a particularly preferred embodiment of the present invention, 1,9-dimethylmethylene blue is employed as the phenothiazin-5-ium dye.

In another preferred embodiment, the partial pressure of oxygen in the red blood cell-containing composition is maintained at a level during irradiation with the phenothiazin-5-ium dye and blocking compound such that the amount of active pathogenic contaminants contained in the red blood cell-containing composition is reduced.

The partial pressure of oxygen in the red blood cell-containing composition can be maintained at a sufficient level by any method or instrument which is capable of exposing air or oxygen gas mixtures to thin films of blood to achieve a reproducible partial pressure of oxygen. For example, the red blood cell-containing composition can be agitated, air or oxygen can be injected into a blood bag containing the red blood cell-containing composition or a blood oxygenator instrument can be used in the inventive method. Pure oxygen or any oxygen gas mixture, e.g., ambient air, may be used in the inventive method provided that the partial pressure of oxygen in the red blood cell-containing composition is maintained at a level during the irradiating procedure such that the amount of active pathogenic contaminants contained in the red blood cell- containing composition is reduced.

In a preferred embodiment, the partial pressure of oxygen in the red blood cell-containing composition is maintained at a level of at least about 40 mm Hg during the irradiating of the phenothiazin-5-ium dye, blocking compound and red blood cell-containing composition, more preferably between about 40 mm Hg and about 600 mm Hg. In a particularly preferred embodiment, the oxygen partial pressure is maintained at a level between about 200 mm Hg and about 550 mm Hg.

The mixture of the red blood cell-containing composition, blocking compound and phenothiazin-5-ium dye is then irradiated with light of an appropriate wavelength (or mixture of wavelengths) and intensity. The term “appropriate wavelength and intensity” is intended to mean light of a wavelength and intensity that can be absorbed by the dye, but does not substantially damage the red blood cells present. It is well within the level of skill in the art to select such wavelength and intensity empirically based on certain relevant parameters, such as the particular dye employed and its concentration in the composition. For example, one having skill in the art would know that if the intensity of the light source is decreased, a greater concentration of dye and/or longer exposure time should probably be used.

An appropriate wavelength is preferably selected based on the absorption profile of the dye (or dyes) employed and is most preferably one that does not result in substantial damage to one or more of the cellular blood components in the composition being decontaminated.

Model viral systems are known to those of skill in the art which may be used to test the selected dye and light source for their efficacy. Such model viral systems include, but are not limited to, the enveloped bacteriophage Φ6, vesicular stomatitis virus (an animal virus that contains its genome encoded in RNA), and Pseudorabies virus (an animal virus that contains its genome encoded as DNA). Based on the effective values of parameters such as wavelength and light intensity measured for such model systems, one of skill in the art can routinely select suitable values for these parameters for use in practice of the present invention.

In a preferred embodiment of this invention, red blood cells, which have optionally been leukodepleted with a five log filter, are first suspended in RAS-2 (Erythrosol) at a hematocrit of about 15% to 65%, then introduced into gas permeable blood preservation bags in an amount such that the filled bag has a thickness of about 1 mm to about 4 mm. Quinacrine is added at a final concentration of about 350 μM to about 750 μM, riboflavin is added at a final concentration of about 15 μM to about 40 μM or lumichrome is added at a final concentration of about 300 μM to about 3 mM. 1,9-dimethylmethylene blue is also added at a concentration of about 1 μM up to about 25 μM. The partial pressure of oxygen in this mixture is then increased to a PO₂ between about 200 mm Hg and about 550 mm Hg and irradiated with red light of wavelength of about 560 to 800 nm at a sufficient intensity for a sufficient time, such as about 5.0-6.0 mW/cm² for about 5-20 seconds, to reduce the level of active pathogenic contaminants in the red blood cell and RAS-2 (Erythrosol) solution. Preferably, the active pathogenic contaminants in the treated sample are inactivated to a log₁₀ inactivation of at least about 4, more preferably at least about 6.

A further embodiment of the present invention provides a method of reducing the level of active pathogenic contaminants in a red blood cell-containing composition which comprises adding to the red blood cell-containing composition at least one phenothiazin-5-ium dye in an amount effective to reduce the level of active pathogenic contaminants and at least one blocking compound selected from the group consisting of quinacrine, riboflavin and lumichrome, or derivatives thereof, and irradiating the red blood cell-containing composition with light of an appropriate intensity and wavelength for a time sufficient to reduce the level of active pathogenic contaminants in the red blood cell-containing composition.

Another embodiment of the present invention provides an irradiated red blood cell-containing composition comprising red blood cells, a phenothiazin-5-ium dye and a blocking compound, wherein the concentration of the blocking compound is in an amount effective to enhance pathogen inactivation and to reduce hemolysis from red blood cells undergoing photoinactivation in the presence of the phenothiazin-5-ium dye.

Following treatment in accordance with the method of this invention, the red blood cell-containing composition may be stored or transfused. If stored, preferably less than about 1% of the red blood cells undergo hemolysis after six weeks of storage at about 1 to about 6° C.

If desired, the phenothiazin-5-ium dye and/or the blocking compound can be removed after treatment. Any suitable method known to those skilled in the art can be used to remove these compounds. For example, after treatment the composition can be centrifuged at a force sufficient to pellet the cellular components. The supernatant can be removed following centrifugation and the cells resuspended to reduce the concentration of residual phenothiazin-5-ium dye and any reaction products.

The following examples are illustrative only and are not intended to limit the scope of the invention as defined by the appended claims. It will be apparent to those skilled in the art that various modifications and variations can be made in the methods of the present invention without departing from the spirit and scope of the invention. Thus, it is intended that the present invention cover the modifications and variations of this invention provided they come within the scope of the appended claims and their equivalents.

All patents and publications referred to herein are expressly incorporated by reference.

### EXAMPLES

### Example 1

### VSV Inactivation in the Presence of Quinacrine

Citrate-phosphate-dextrose anticoagulated whole blood was centrifuged at room temperature (Sorval centrifuge using P1 program setting). Platelet rich plasma and 50 g of buffy coat were discarded. 100 ml of the additive solution RAS-2 (Erythrosol) was added to the packed red cells. The packed red cells (55-70% hematocrit) were filtered through a Pall BPF-4 filter at room temperature to remove leukocytes and subsequently chilled to 1-6° C. Hematocrit was measured. 56.3 ml of red blood cells were mixed with 8 ml of chilled vesicular stomatitis virus (VSV) and the hematocrit for this mixture was 56.04%.

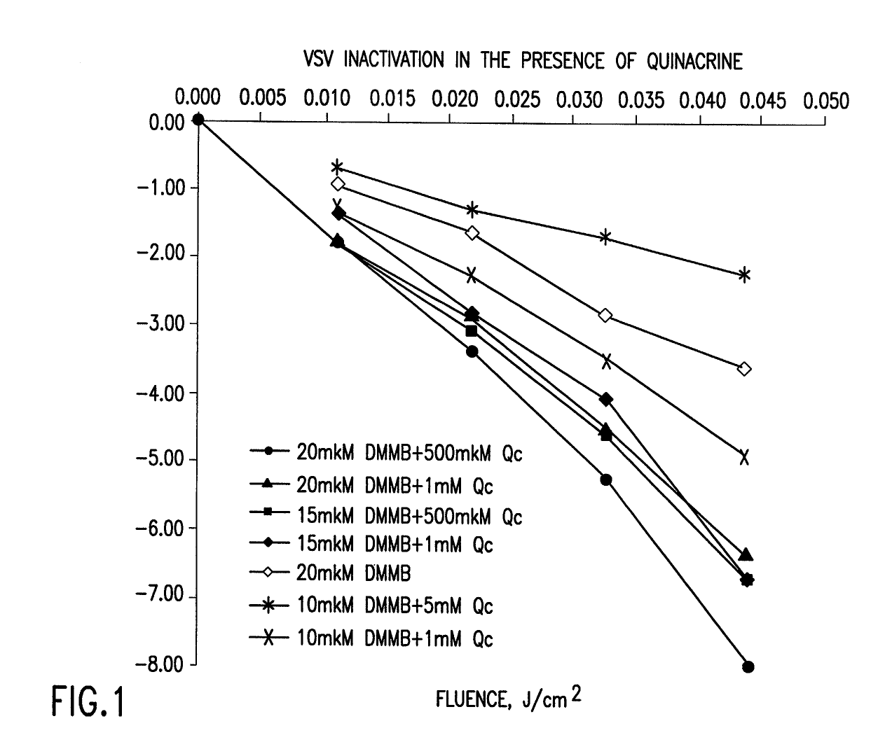

For not treated (NT) samples, 1.61 ml of red blood cells and VSV were mixed with 0.39 ml of RAS-2 (Erythrosol). For experimental samples, 8 ml of red blood cell/VSV suspension was added to 1.75 ml of chilled RAS-2 (Erythrosol) to make a final hematocrit of about 46%. Further addition of quinacrine and dimethylmethylene blue (DMMB) brought the final hematocrit to 45%. Quinacrine was added into the red blood cell-VSV mixture 15 minutes before DMMB. After DMMB addition, blood was dispensed into Petri dishes in 2 ml aliquots, agitated for 15 minutes at 4° C. (pO₂=305.7 mm Hg) and treated with light (Q-Beam LED, Quantum Devices). Samples were titrated for virus and plaques were stained on the next day. The results are presented in FIG. 1.

### Example 2

### VSV Inactivation in the Presence of Quinacrine

Citrate-phosphate-dextrose anticoagulated whole blood was centrifuged at room temperature (Sorval centrifuge using P1 program setting). Platelet rich plasma and 50 g of buffy coat were discarded. 100 mL of the additive solution RAS-2 (Erythrosol) was added to the packed cells. The packed cells (55-70% hematocrit) were filtered through a Pall BPF-4 filter at room temperature to remove leukocytes and subsequently chilled to 1-6° C. Hematocrit was measured (64%). 10.5 mL of red blood cells were mixed with 1.5 mL of vesicular stomatitis virus (VSV) and 75 μL of 100 mM quinacrine. After 15 minutes incubation on ice, 2.85 mL of RAS-2 and 75 μL of DMMB was added to bring the hematocrit to 45% and the DMMB concentration to 5 μM. After DMMB addition, blood was dispensed into Petri dishes in 2 mL aliquots, agitated for 15 minutes at 4° C. (pO2=303.4 mm Hg) and subsequently treated with light (Q-Beam LED, Quantum Devices). Samples were titrated for virus and plaques were stained on the next day. Results are presented in the following table:

| Fluence (mJ/cm²) | Log₁₀ VSV inactivation |
|---|---|
| 0 | 0.0 |
| 27 | 1.9 |
| 54 | 3.9 |
| 81 | 6.2 |
| 108 | >7.5 |
| 135 | >7.5 |

### Example 3

### Effect of Quinacrine on Red Cell Hemolysis During Storage

The effects of quinacrine on the hemolysis of 1,9-dimethylmethylene blue and light treated red blood cells was studied. Quinacrine was added to the red blood cell-containing composition at a final concentration of 500 μM, and DMMB was added at a final concentration of either 5 μM, 15 μM, or 20 μM. The mixture was irradiated for either 9 seconds or 20 seconds with red light (LED), 0.0486 J/cm² or 0.108 J/cm², respectively. Based on the VSV inactivation experiments presented in Examples 1 and 2,>6 log₁₀ inactivation should be achieved for each of the phototreated samples.

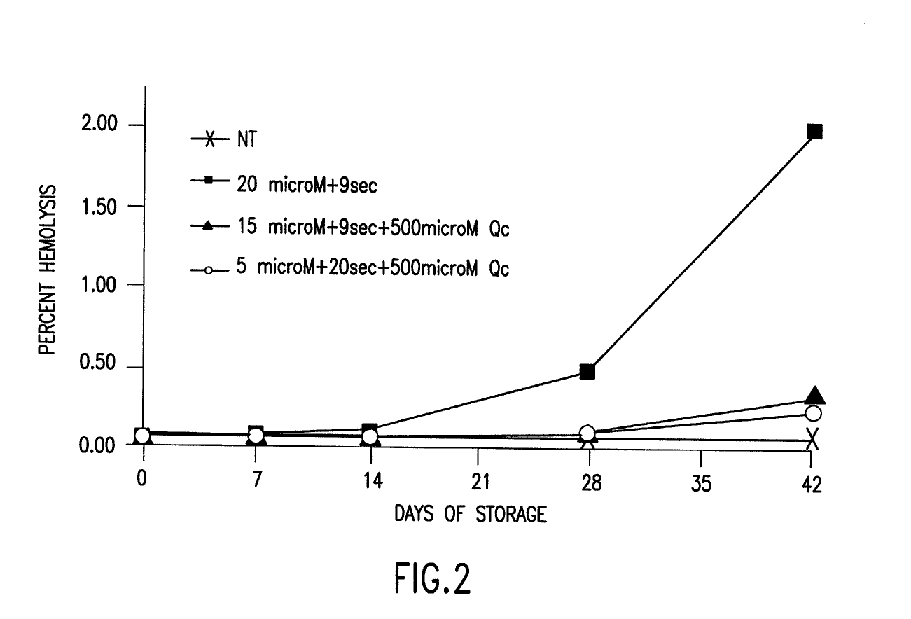

As shown in FIG. 2, samples treated with DMMB and quinacrine exhibited less than 0.5% hemolysis after 42 days storage at 1-6° C.

### Example 4

### VSV Inactivation in the Presence of Riboflavin

Citrate-phosphate-dextrose anticoagulated whole blood was centrifuged at room temperature (Sorval centrifuge using P1 program setting). Platelet rich plasma and 50 g of buffy coat were discarded. 100 ml of the additive solution RAS-2 (Erythrosol) was added to the packed red cells. The packed red cells (55-70% hematocrit) were filtered through a Pall BPF-4 filter at room temperature to remove leukocytes. Hematocrit was measured. Red blood cells were mixed with an adequate volume of RAS-2 (Erythrosol) and riboflavin following VSV addition to bring the hematocrit down to 45% and were incubated on ice for 15 minutes. DMMB was added to a final concentration of 5 μM.

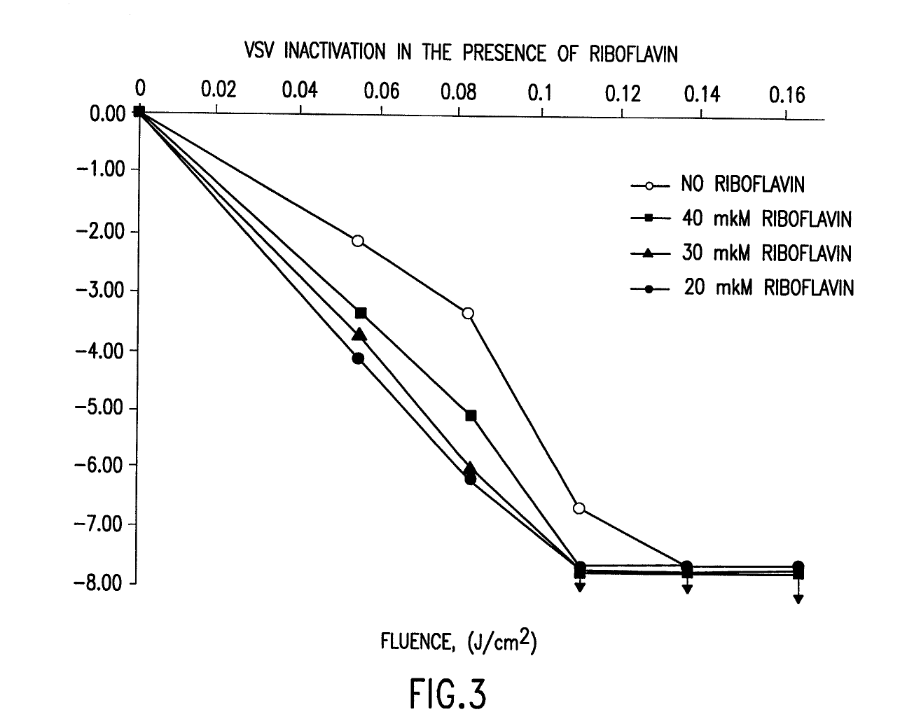

Samples were dispensed into Petri dishes (1 mm sample) and agitated at 1-6° C. for 15 minutes at 250 rpm (pO₂=306.3 mm Hg). Riboflavin was freshly prepared and the samples were titrated for virus immediately after irradiation. Plaques were stained on the following day. The results are shown in FIG. 3.

### Example 5

### VSV Inactivation and Red Cell Hemolysis for DMMB+Light Treated Samples (no blocker present) in a Flow System

Citrate-phosphate-dextrose anticoagulated whole blood was leukodepleted at room temperature (Sepacell RS2000 filter) and subsequently centrifuged at 2800 rpm for 4 minutes at 4° C. (Sorval RC-3C centrifuge). The supernatant was discarded and 90 mL of 1-6° C. RAS-2 (Erythrosol) was added to the packed cells. The hematocrit of the packed cells was measured. Red cells were mixed with an adequate volume of RAS-2 and agitated for 20 minutes at 1-6° C. to give a final hematocrit of 50.7% and a pO₂ of 439.6 mm Hg.

The following additions were made to the red cell suspension in the indicated order:

| | Control | Treated |
|---|---|---|
| Additions | (sample #1) | (for samples #2, 3, 4 and 5) |
| RBC, mL | 52.9 | 222 |
| RAS-2, mL | 7.1 | 28.5 |
| VSV, mL | None | 9 |
| DMMB mL (720 μM) | None | 1.26 |
| Total | 60 | 260.76 |

These additions gave a final hematocrit of approximately 45% and a final DMMB concentration of approximately 3.5 μM. Treated samples were aliquoted into 60 mL samples in blood bags, incubated at 1-6° C. for 15 minutes, and sterile connected to a flow cell device (with 1 mm optical path length) with receiving bag. Samples #2, 3, 4 and 5 were illuminated with 0, 226, 889 or 1597 mJ/cm² red (LED) light in the flow system. VSV was titered and plaques were stained on the following day. Control and phototreated samples were stored at 1-6° C. and assayed for hemolysis at day 42. Results are depicted in the following table:

| Sample | Light fluence | VSV Inactivation | Hemolysis (%), |
|---|---|---|---|
| # | (mJ/cm²) | (log₁₀) | day 42 |
| 1 | 0 | 0.0 | 0.07 |
| 2 | 0 | 0.0 | Not performed |
| 3 | 226 | 2.2 | 2.84 |
| 4 | 889 | 3.4 | 14.53 |
| 5 | 1597 | 3.6 | 12.10 |

### Example 6

### VSV Inactivation and Red Cell Hemolysis for DMMB+Light Treated Samples (containing riboflavin as a blocker) in a Flow System

Citrate-phosphate-dextrose anticoagulated whole blood was leukodepleted at room temperature (Sepacell RS2000 filter) and subsequently centrifuged at 2800 rpm for 4 minutes at 4° C. (Sorval RC-3C centrifuge). The supernatant was discarded and 90 mL of 1-6° C. RAS-2 (Erythrosol) was added to the packed cells. The hematocrit of the packed cells was measured. Red cells were mixed with an adequate volume of RAS-2 and agitated for 20 minutes at 1-6° C. to give a final hematocrit of 50.7% and a pO₂ of 536.2 mm Hg.

The following additions were made to the red cell suspension in the indicated order:

| Additions | Control | Treated |
|---|---|---|
| RBC, mL | 52.3 | 69.8 |
| RAS-2, mL | 7.7 | 5.4 |
| VSV, mL | | 4 |
| Riboflavin mL (4 mM) | | 0.4 |
| DMMB mL (720 μM) | | 0.4 |
| Total | 60 | 80 |

These additions gave a final hematocrit of approximately 45%, and for the treated sample, a final riboflavin concentration of approximately 20 μM, and a final DMMB concentration of approximately 3.6 μM. Samples were incubated at 1-6° C. for 15 minuted after riboflavin and DMMB additions, respectively, and subsequently sterile connected to a flow cell device (1 mm optical path length) with receiving bag. Treated samples were then illuminated with 0 or 226 mJ/cm² red (LED) light in the flow system. VSV was titered and plaques were stained on the following day. Control and phototreated samples were stored at 1-6° C. and assayed for hemolysis at day 42. Results are depicted in the following table:

| | Light fluence | VSV Inactivation | Hemolysis (%), |
|---|---|---|---|
| Sample | (mJ/cm²) | (log₁₀) | day 42 |
| Control | 0 | 0.0 | 0.07 |
| Treated | 226 | >6.3 | 1.44 |

Based on VSV inactivation and hemolysis results from Examples 4 and 5, it is evident that the addition of the blocker, riboflavin, to treatment of 45% Hct red cells with about 3.5 to about 3.6 μM DMMB and about 226 J/cm² red (LED) light enhances VSV inactivation by >4.1 log₁₀ and reduces hemolysis at day 42 by approximately a factor of 2. In the absence of riboflavin, attempts to achieve greater VSV inactivation by illuminating samples lacking riboflavin with greater light fluences results in only a 50% increase in inactivation (2.2 to 3.4 log₁₀) yet generates a 500% increase in hemolysis (2.8 to 14.5%).

What is claimed is:

1. A method of reducing the level of active pathogenic contaminants present in a red blood cell-containing composition which comprises:
(a) adding to said red blood cell-containing composition at least one phenothiazin-5-ium dye in an amount effective to reduce the level of active pathogenic contaminants and at least one blocking compound in an amount effective to enhance pathogen inactivation and protect red blood cells from hemolysis; and
(b) irradiating said red blood cell-containing composition with light of an appropriate intensity and wavelength for a time sufficient to reduce the level of said active pathogenic contaminants in said red blood cell-containing composition.

2. The method of claim 1, wherein said blocking compound comprises a fused aromatic ring system containing at least three rings, wherein at least one ring contains at least one heteroatom.

3. The method of claim 2, wherein said blocking compound comprises an acridine moiety, an alloxazine moiety, a phenothiazine moiety, a phenoxazine moiety or a phenazine moiety.

4. The method of claim 3, wherein said blocking compound comprises quinacrine or a derivative thereof.

5. The method of claim 3, wherein said blocking compound comprises riboflavin or a derivative thereof.

6. The method of claim 3, wherein said blocking compound comprises lumichrome or derivative thereof.

7. The method of claim 4, wherein said effective amount of said blocking compound is an amount sufficient to provide a concentration of said blocking compound in said red blood cell-containing composition from about 250 μM to about 1000 μM.

8. The method of claim 7, wherein said effective amount of said blocking compound is an amount sufficient to provide a concentration of said blocking compound in said red blood cell-containing composition from about 350 μM to about 750 μM.

9. The method of claim 5, wherein said effective amount of said blocking compound is an amount sufficient to provide a concentration of said blocking compound in said red blood cell-containing composition from about 5 μM to about 45 μM.

10. The method of claim 9, wherein said effective amount of said blocking compound is an amount sufficient to provide a concentration of said blocking compound in said red blood cell-containing composition from about 15 μM to about 40 μM.

11. The method of claim 6, wherein said effective amount of said blocking compound is an amount sufficient to provide a concentration of said blocking compound in said red blood cell-containing composition from about 300 μM to about 3 mM.

12. The method of claim 1, wherein said red blood cell-containing composition is whole blood or a red blood cell concentrate.

13. The method of claim 1, wherein said active pathogenic contaminants in said red blood cell-containing composition are inactivated to a log₁₀ inactivation of at least about 4.

14. The method of claim 13, wherein said active pathogenic contaminants in said red blood cell-containing composition are inactivated to a log₁₀ inactivation of at least about 6.

15. The method of claim 1, further comprising leukodepleting said red blood cell-containing composition prior to the adding of (a).

16. The method of claim 1, wherein said appropriate wavelength is between about 500 nm and about 800 nm.

17. The method of claim 16, wherein said appropriate intensity is between about 0.2 mW/cm² and about 10.0 mW/cm².

18. The method of claim 17, wherein the fluence is from about 1 mJ/cm² to about 5 00 mJ/CM².

19. The method of claim 17, wherein the fluence is from about 1 mJ/cm² to about 120 mJ/cm².

20. The method of claim 1, wherein the partial pressure of oxygen in said red blood cell-containing composition is maintained at a level between about 40 mm Hg and about 600 mm Hg during the irradiating of said red blood cell-containing composition.

21. The method of claim 20, wherein the partial pressure of oxygen in said red blood cell-containing composition is maintained at a level between about 200 mm Hg and about 550 mm Hg during the irradiating of said red blood cell-containing composition.

22. The method of claim 1, wherein said effective amount of said phenothiazin-5-ium dye is an amount sufficient to provide a concentration of said phenothiazin-5-ium dye in said red blood cell-containing composition from about 0.2 μM to about 50 μM.

23. The method of claim 1, wherein said phenothiazin-5-ium dye is 1,9-dimethylmethylene blue.

24. The method of claim 1, wherein said red blood cell-containing composition is transfusable.

25. The method of claim 1, wherein said pathogenic contaminants are selected from the group consisting of viruses, bacteria, parasites and leukocytes.

26. The method of claim 25, wherein said viruses are selected from the group consisting of intracellular viruses and extracellular viruses.

27. The method of claim 25, wherein said viruses are enveloped viruses.

28. The method of claim 27, wherein said enveloped viruses include at least one virus selected from the group consisting of RNA viruses and DNA viruses.

29. The method of claim 28, wherein said enveloped viruses include at least one or more viruses selected from the group consisting of human immunodeficiency virus, cytomegalovirus, herpes virus, hepatitis A virus, hepatitis B virus, hepatitis C virus, Vesicular Stomatitis virus, Sindbis virus and pox virus.

30. The method of claim 25, wherein said bacteria include members of the genus Streptococcus or the genus Escherichia.

31. The method of claim 25, wherein said parasites include members of the genus Trypanosoma.

32. A method of reducing the level of active pathogenic contaminants present in a red blood cell-containing composition which comprises:
(a) adding to said red blood cell-containing composition at least one phenothiazin-5-ium dye in an amount effective to reduce the level of active pathogenic contaminants and at least one blocking compound selected from the group consisting of quinacrine, riboflavin and lumichrome, and derivatives thereof, in an amount effective to enhance pathogen inactivation and protect red blood cells from hemolysis; and
(b) irradiating said red blood cell-containing composition with light of an appropriate intensity and wavelength for a time sufficient to reduce the level of said active pathogenic contaminants in said red blood cell-containing composition.

33. The method of claim 32, wherein said blocking compound is quinacrine or a derivative thereof.

34. The method of claim 32, wherein said blocking compound is riboflavin or a derivative thereof.

35. The method of claim 32, wherein said blocking compound is lumichrome or derivative thereof.

36. The method of claim 33, wherein said effective amount of said blocking compound is an amount sufficient to provide a concentration of said blocking compound in said red blood cell-containing composition from about 250 μM to about 1000 μM.

37. The method of claim 36, wherein said effective amount of said blocking compound is an amount sufficient to provide a concentration of said blocking compound in said red blood cell-containing composition from about 3 50 μM to about 750 μM.

38. The method of claim 34, wherein said effective amount of said blocking compound is an amount sufficient to provide a concentration of said blocking compound in said red blood cell-containing composition from about 5 μM to about 45 μM.

39. The method of claim 38, wherein said effective amount of said blocking compound is an amount sufficient to provide a concentration of said blocking compound in said red blood cell-containing composition from about 15 μM to about 40 μM.

40. The method of claim 35, wherein said effective amount of said blocking compound is an amount sufficient to provide a concentration of said blocking compound in said red blood cell-containing composition from about 300 μM to about 3 mM.

41. A method of enhancing pathogen inactivation in a red blood cell-containing composition which comprises:
(a) adding to said red blood cell-containing composition at least one phenothiazin-5-ium dye in an amount effective to reduce the level of active pathogenic contaminants and at least one blocking compound selected from the group consisting of quinacrine, riboflavin and lumichrome, and derivatives thereof, in an amount effective to enhance pathogen inactivation; and
(b) irradiating said red blood cell-containing composition with light of an appropriate intensity and wavelength for a time sufficient to reduce the level of said active pathogenic contaminants in said red blood cell-containing composition,
wherein said level of pathogen inactivation is enhanced as compared to the level of pathogen inactivation from phenothiazin-5-ium dye phototreatment without said blocking compound.

42. The method of claim 41, wherein said blocking compound is quinacrine or a derivative thereof.

43. The method of claim 41, wherein said blocking compound is riboflavin or a derivative thereof.

44. The method of claim 41, wherein said blocking compound is lumichrome or a derivative thereof.

45. A method of protecting a red blood cell-containing composition from phenothiazin-5-ium dye and light induced hemolysis which comprises:
(a) adding to said red blood cell-containing composition at least one phenothiazin-5-ium dye in an amount effective to reduce the level of active pathogenic contaminants and at least one blocking compound selected from the group consisting of quinacrine, riboflavin and lumichrome, and derivatives thereof, in an amount effective to protect red blood cells from hemolysis; and
(b) irradiating said red blood cell-containing composition with light of an appropriate intensity and wavelength for a time sufficient to reduce the level of said active pathogenic contaminants in said red blood cell-containing composition,
wherein said level of hemolysis from phenothiazin-5-ium dye phototreatment is reduced as compared to hemolysis levels ofphenothiazin-5-ium dye phototreatment without said blocking compound.

46. The method of claim 45, wherein said blocking compound is quinacrine or a derivative thereof.

47. The method of claim 45, wherein said blocking compound is riboflavin or a derivative thereof.

48. The method of claim 45, wherein said blocking compound is lumichrome or derivative thereof.

49. An irradiated red blood cell-containing composition comprising red blood cells, a phenothiazin-5-ium dye and a blocking compound, wherein the concentration of said blocking compound is in an amount effective to enhance pathogen inactivation and to reduce hemolysis from red blood cells undergoing photoinactivation in the presence of said phenothiazin-5-ium dye.

50. The composition of claim 49, wherein said blocking compound comprises a fused aromatic ring system containing at least three rings, wherein at least one ring contains at least one heteroatom.

51. The composition of claim 50, wherein said blocking compound comprises an acridine moiety, an alloxazine moiety, a phenothiazine moiety, a phenoxazine moiety or a phenazine moiety.

52. The composition of claim 51, wherein said blocking compound is quinacrine or a derivative thereof.

53. The composition of claim 51, wherein said blocking compound is riboflavin or a derivative thereof.

54. The composition of claim 51, wherein said blocking compound is lumichrome or a derivative thereof.

55. The composition of claim 52, wherein said effective amount of said blocking compound is an amount sufficient to provide a concentration of said blocking compound in said red blood cell-containing composition from about 250 μM to about 1000 μM.

56. The composition of claim 55, wherein said effective amount of said blocking compound is an amount sufficient to provide a concentration of said blocking compound in said red blood cell-containing composition from about 350 μM to about 750 μM.

57. The composition of claim 53, wherein said effective amount of said blocking compound is an amount sufficient to provide a concentration of said blocking compound in said red blood cell-containing composition from about 5 μM to about 45 μM.

58. The composition of claim 57, wherein said effective amount of said blocking compound is an amount sufficient to provide a concentration of said blocking compound in said red blood cell-containing composition from about 15 μM to about 40 μM.

59. The composition of claim 54, wherein said effective amount of said blocking compound is an amount sufficient to provide a concentration of said blocking compound in said red blood cell-containing composition from about 300 μM to about 3 mM.

60. The composition of claim 49, wherein said red blood cell-containing composition is whole blood or a red blood cell concentrate.

61. The composition of claim 49, wherein said phenothiazin-5-ium dye is 1,9-dimethylmethylene blue.
